(19) United States
(12) Patent Application Publication
(10) Pub. No.: US 2025/0092354 A1
(43) Pub. Date: Mar. 20, 2025

(54) METHOD FOR PRODUCING BACTERIA

(71) Applicant: Chr. Hansen A/S, Hoersholm (DK)

(72) Inventors: Carina SVENDSEN, Hoersholm (DK); Tina Malling THORSEN, Hoersholm (DK); Vojislav VOJINOVIC, Hoersholm (DK)

(73) Assignee: Chr. Hansen A/S, Hoersholm (DK)

(21) Appl. No.: 18/726,697

(22) PCT Filed: Jan. 10, 2023
(86) PCT No.: PCT/EP2023/050457

§ 371 Date: Jul. 3, 2024

(30) Foreign Application Priority Data
Jan. 11, 2022 (EP) 22150926.8

(51) Int. Cl. C12N 1/20; A23L 2/02; A23L 2/52; C12N 1/04

(52) U.S. Cl. CPC C12N 1/20; A23L 2/02; A23L 2/52; C12N 1/04; A23V 2400/175

(57) ABSTRACT

The present invention relates to methods for producing bacteria, designed to enhance the bacteria by subjecting them to stress conditions. The invention also relates to use of the produced bacteria for the preparation of food products.

### FIELD OF INVENTION

The present invention relates to methods for producing bacteria, including subjecting the bacteria to stress conditions and the use of these bacteria for the preparation of food products, in particular fruit food products having low pH.

### BACKGROUND OF INVENTION

Some bacteria are known to have beneficial health effect on the host when ingested in adequate viable amounts. Particularly, bacterial strains belonging to the genera Lactobacillus and Bifidobacterium have been the subject of many studies demonstrating preventive clinical effects in various fields and on certain physiological functions. These probiotics are generally safe and notably capable of promoting proper operation of the intestinal flora. Besides clear evidence that it has positive effects on the health of the consumer, for a bacterium to fulfil the probiotic definition, it has to be able to survive in and colonize the intestines and survive the harsh processes at production and storage of the food. The clinical evidence indicates that the daily dose of probiotic bacteria should be at least 10⁹ CFU to ensure probiotic efficacy.

Therefore, for a number of years, agro-food industries have attempted to incorporate such bacteria in their final food products, such as beverages. Food products are an excellent way of administrating bacteria to the consumer.

Methods for producing a food product containing bacteria cultures are known in the art, such as in European Patent 0 113 055, which is related to a method comprising bringing a fruit juice into contact with a solid agent to remove the bacteriostatic components and then proliferating Lactobacillus in the fruit juice at a pH of 4.0 or lower. However, in such fruit beverages it is possible to observe bacterial growth and/or activity which induce a production of gas and off-flavor which makes them unsuitable for consumption.

US Patent Application US 2010/0086646 relates to fresh plant juice and/or milk-based food product comprising live probiotics and a dietary protonated weak monoacid with a pH between 3 and 4, the latter prevented probiotics from producing false taste and/or gas in the food product. The addition of monoacids may affect the organoleptic properties of the food product and has also been seen to affect negatively the survival rate of the probiotic bacteria. The addition of acid formation reducers also seems to negatively affect the survival rate of probiotic bacteria. Addition of agents for removal of bacteriostatic compounds and/or for reducing false taste and/or gas in the food product may furthermore prove to be costly.

US Patent Application US 2008/0026102 discloses food additive comprising bacteria concentrates in liquid form comprising adapted and viable bacteria at a concentration between 5×10¹⁰ and 5×10¹¹ ufc/ml, said adapted and viable bacteria being more resistant to various physiochemical stresses. The bacteria may be adapted using a step of natural acidification.

European Patent 2 665 378 discloses a method for producing a probiotic fruit beverage comprising the step of acid-adapting at least one strain of probiotic bacteria during propagation.

There remains a need within the technical field to provide alternative methods for producing high yields of bacteria, in particular probiotics, which have a high viability in acid environments. These cultures are needed for the production of food products with a high amount of live cells, wherein the food product exhibits a good taste without off-flavor and gas production, and wherein the food product has a long shelf-life. By “off-flavor” is meant an abnormal taste of the food product. The off-flavor is unpleasant for the consumer and therefore not sought.

### SUMMARY OF THE INVENTION

Accordingly, an object of the present invention is to increase the yields and provide high amounts of robust bacteria capable of surviving the acidic environment of some beverages. It is a further object of the present invention to provide a food product, such as a beverage, containing live bacteria with a good taste. Yet another object of the present invention is to provide such a beverage which has a long shelf-life.

When incorporating bacteria into a food product, it is important that the bacteria is adapted to the environment of the food product. When the food product is acidic, such acidic environments cause a large initial cell count reduction when a reference bulk is added. For successful application of bacteria cultures in food products, appropriate concentrations of cells must be ensured during shelf life. It would therefore be desirable to produce high amounts of bacteria having a maximum viability and activity in food products having acidic pH.

According to the present invention the above problems are solved by a method for producing bacteria, said method comprising the steps of:
(a) inoculating bacteria in a medium which medium is acidified until a first pH between 3 and 7 is reached;
(b) cultivating the bacteria in the medium while maintaining the first pH by adding an alkaline solution to the medium;
(c) allowing the medium to acidify until a second pH is reached wherein the second pH is lower than the first pH; and
(d) harvesting the bacteria.

The present invention proposes an advantageous method for producing bacteria for use in food products, such as beverages. The present invention is based on the finding that bacteria may be adapted to acid by propagation without pH stabilization for a certain period of time without significantly affecting the resulting yield in fermentation.

Surprisingly and unexpectedly, the inventors showed that a step of adapting the bacteria made it possible to significantly increase the viability of the bacteria in a food product. This has extended the type of beverage which can be prepared. The cultures of bacteria strains of the present invention are extremely useful as compositions for the addition to beverages with a low pH (below 4.2).

The method of the present invention allows increased biomass yields and good survival in low pH beverages, in particular juices and similar products. Acid stress during production is believed to prepare the cells for survival in harsh environment characterized by low pH, but also other stress factors.

The increase in biomass yield leads to a more efficient process, which is more sustainable and financially appealing.

According to a second aspect, the present invention provides bacteria obtainable by the method according to the first aspect.

A third aspect of the present invention concerns the use of a culture of a bacteria strain according to the second aspect for the production of a food product.

A fourth aspect relates to a method of producing a food product comprising the steps of:
(i) producing bacteria according to the method of any of claims 1 to 11,
(ii) adding the bacteria to the food product; and
(iii) optionally packaging the food product.

A fifth aspect concerns a food product obtainable by the method according to the fourth aspect of the invention, wherein the bacteria is present in the food product in an amount from 1×10³ to 1×10¹⁰ CFU/ml, 1×10⁴ to 1×10⁹ CFU/ml, or 1×10⁵ to 1×10⁸ CFU/ml after 70 days of storage at a temperature between 2 and 10° C.

### DETAILED DESCRIPTION OF THE INVENTION

According to the present invention methods for producing bacteria are provided, which comprise the steps of:
(a) inoculating bacteria in a medium which medium is acidified until a first pH between 3 and 7 is reached;
(b) cultivating the bacteria in the medium while maintaining the first pH by adding an alkaline solution to the medium;
(c) allowing the medium to acidify until a second pH is reached wherein the second pH is lower than the first pH; and
(d) harvesting the bacteria.

In the context of the present application the term “method for producing bacteria” refers to methods of culturing and propagating bacteria for example by fermentation in order to obtain a bacterial culture. The method may additionally comprise steps of concentrating the bacteria after propagation.

The method of the present invention can comprise the following four steps: a) the bacteria are allowed to grow and metabolize the medium which causes acidification. When a defined pH between 3 and 7 is reached, step b) can be initiated, wherein alkaline solution is added to keep the fermentation medium pH at this constant value. The addition of the alkaline solution can balance the acidification of the medium due to the production of acids by the bacteria. After a selected time, the step c) can be initiated, wherein the addition of base is terminated, and the pH is allowed to reach a lower pH. At this selected pH, the method can be terminated and cells can be harvested. The pH can be monitored and controlled during the whole process.

In one embodiment the at least one bacteria is grown in step a) at a temperature in the range from 20° C. to 45° C., preferably in the range from 25° C. to 43° C., such as 37° C. to 43° C.

In a preferred embodiment, the acidification in step a) is achieved by natural acidification. Bacteria cause a drop in the pH during propagation due to the production of lactic acid during fermentation. In this case, no external acid is added to the medium.

In another embodiment, the acidification in step a) may also be achieved (in part or in full) by the continuous or stepwise addition of acid to the culture.

The medium used in the method of the present invention preferably has a composition which allows said medium to reach a low pH during propagation of the bacteria. This means that the pH of the medium decreases due to the production of acid by the bacteria, or, alternatively, by a continuous or stepwise addition of acid to the culture. Accordingly, the medium does not contain any essential components which would decompose or otherwise become ineffective or even toxic at a low pH. Essential components of the medium are compounds that are required for the propagation of bacteria, such as nutrients, carbon and nitrogen sources, vitamins and the like.

In one embodiment of the present invention, the medium which is inoculated with the bacteria does not contain buffering agents that could keep the pH stable upon the formation or addition of acid during propagation. Alternatively, where the starting medium contains a certain concentration of buffering agents, culturing can be continued until the capacity of the buffering agent is exhausted, and no additional buffering agents are added during culturing. In a preferred embodiment, the medium contains less buffering agents than a standard MRS medium (as formulated in J. C de Man et. al, (1960), J. appl. Bad. 23 (1), 130-135).

The initial pH of the medium is the pH of the medium prior to addition of bacteria. In a preferred embodiment the initial pH of the medium is between about 6.0 and 7.0, preferably between about 6.2 and about 6.6.

In one embodiment of the present invention, the medium in step a) is allowed to acidify until a first pH between 3.5-6.5, 4.0-6.0, 4.5-5.5, 4.6-5.4, 4.7-5.3, 4.8-5.2, 4.9-5.1, or is 4.0, 4.1, 4.5, 4.6, 4.7, 4.8, 4.9, 5.0, 5.1, 5.2, 5.3, 5.4, 5.5 is reached.

In one embodiment of the present invention, the medium in step a) is allowed to acidify until a pH between 5.5 and 4.5, preferably between 4.9 and 5.2 is reached.

Preferably the defined pH is reached in step a) after not more than 18, such as between 3 and 12 hours or 4 to 7 hours. This time is measured from the time the reactor is inoculated and warm, until the first pH is reached.

After the medium has reached the defined pH, the pH of the medium is maintained at the first pH in step b) for a defined time. In one embodiment, the first pH of the medium in step b) is maintained for a period of 1-24 hours, 3-22, 5-20, 7-18, 8-16, 9-14, or 6, 7, 8, 9, 10, 11, 12, 13, 14, 15 hours.

In one embodiment, the pH of the medium in step b) is maintained at the first pH level for at least 1 hour, such as 2 hours or 3 hours, by adding an alkaline solution to the medium. In a particularly preferred embodiment, the pH of the medium is maintained at the defined pH for a time of at least 4 hours.

In an embodiment of the invention, the pH of the medium is maintained at the defined pH in step b) for a time between 1 and 24 hours. Preferably, the pH of the medium is maintained at the defined pH for a time between 3 and 22 hours, such as 4 and 20 hours. In a particularly preferred embodiment, the pH of the medium is maintained at the defined pH for a time between 4 and 10 hours, such as 5 to 8 or 6 to 7 hours.

In a preferred embodiment of the invention the pH of the medium in step b) is maintained by incremental addition of alkaline solution. In a particularly preferred embodiment the pH of the medium in step b) is maintained by incremental addition of alkaline solution while monitoring the pH of the medium.

The term “alkaline solution” refers to a solution of a soluble base having a pH greater than 7.0. The pH of the alkaline solution may be greater than 7.5, greater than 8, or greater than 9. The alkaline solution may be an aqueous solution of a basic salt, in particular an alkali metal hydroxide, alkaline earth metal hydroxide or ammonium hydroxide. Preferably, aqueous solution of sodium hydroxide or ammonium hydroxide are used.

After the pH has been maintained for the defined time, the medium is allowed to acidify until a second pH is reached which is even more acidic. In one embodiment, the second pH is between 2.0-6.0, 2.5-6.0, 3.0-6.0, 3.5-5.5, 4.0-5.0, 4.1-4.9, 4.2-4.8, 4.3-4.7, 4.4-4.6, or is pH 4.0, 4.1, 4.2, 4.3, 4.4, 4.5, 4.6, 4.7, 4.8, 4.9 or 5.0.

In a preferred embodiment, the medium in step c) is allowed to reach a second pH value of between 2 and 5, preferably between pH 3.5 and 5.0, more preferably between pH 3.8 and 4.5.

In a preferred embodiment, the acidification in step c) is achieved by natural acidification. The acidification may also be achieved (in part or in full) by the continuous or stepwise addition of acid to the culture.

In a preferred embodiment of the invention the medium reaches the second defined pH after 2 to 15 hours, preferably after 4 to 12 hours, more preferably between 4 and 11 hours.

The total time of steps a) to c) is preferably between 4-24, 6-22, 8-20, 10-18, 12-16, 13-15 hours, or is not more than 24, 22, 20, 18, 16, 15, 14, 13, 12, 10, 8, 6, 4 hours.

In a preferred embodiment, the total time of steps a) to c) is not more than 20 hours such as 4 to 18 hours or 7 to 15 hours.

The at least one strain of bacteria is harvested and is preferably concentrated. Methods for harvesting and concentrating the bacteria are well known in the art. For example, harvesting of the bacteria may be achieved by centrifugation of the bacterial cultures to remove the culture media supernatant. The centrifugation will be gentle enough to avoid breakage of the bacteria. Methods for harvesting cells by centrifugation are commonly known and include, e.g. centrifugation at 2000 to 6000×g, such as 5800×g for 2-20 minutes. The bacteria may also be harvested by other techniques that have been described in the context of bacterial fermentation, such as filtration, e.g. cross-filtration filtration. Methods for concentration are likewise known in the art. For example, concentration of the bacteria may be achieved by centrifugation and subsequent resuspension in a smaller volume, freeze-drying or membrane filtration techniques, such as filtration through columns or filters. If resuspension is used, the cells may be resuspended in re-used original growth medium.

In a preferred embodiment, the bacteria are not washed after being harvested.

Afterwards, the at least one strain of bacteria is optionally frozen or freeze-dried. For example, the bacteria may be frozen at about −20° C., preferably at about −80° C., or lower. The freezing procedure is preferably carried out as quickly as possible, preferably by shock-freezing, to avoid cellular damage. Shock-freezing may e.g. be carried out by dumping a vessel containing the bacteria into liquid nitrogen. Thus, in a particularly preferred embodiment the bacteria are shock frozen at about −196° C. Methods for freeze-drying are also well known in the art. During freeze-drying the material is frozen and then the surrounding pressure is reduced to allow the frozen water in the material to sublimate directly from the solid phase to the gas phase. After freezing or shock-freezing the produced bacteria, the bacteria may be stored at about −20° C., at about −55° C., at about −80° C., or in liquid nitrogen at −196° C. It is particularly preferred to store the bacteria at about −55° C.

Prior to be used in the method of the present invention, the at least one strain of bacteria may be propagated using standard techniques in order to obtain a sufficient amount of bacteria to be used in the method of the present invention. Generally, the skilled person will know of suitable media for the propagation of bacteria.

In a preferred embodiment of the present invention the at least one strain of bacteria is a probiotic bacteria. The term “probiotic bacteria” refers to viable bacteria which are administered in adequate amounts to a consumer for the purpose of achieving a health-promoting effect in the consumer. Probiotic bacteria are capable of surviving the conditions of the gastrointestinal tract after ingestion and colonize the intestine of the consumer. The probiotic bacteria can be of the genus Lactobacillus, such as Lactobacillus acidophilus, Lactobacillus paracasei, Lactobacillus rhamnosus, Lactobacillus casei, Lactobacillus delbrueckii, Lactobacillus lactis, Lactobacillus plantarum, Lactobacillus reuteri, Lactobacillus bulgaricus, Lactobacillus helveticus and Lactobacillus johnsonii. The probiotic bacteria can also be of the genus Bifidobacterium, such as the Bifidobacterium animalis, Bifidobacterium breve, Bifidobacterium infantis, Bifidobacterium longum.

In another preferred embodiment, the at least one strain of bacteria is a Oenococcus oeni bacterium.

In a preferred embodiment of the present invention the at least one strain of bacteria is a Lactobacillus paracasei, Lactobacillus rhamnosus, Lactobacillus casei or Bifidobacterium animalis ssp. lactis strain.

In a more preferred embodiment of the present invention the at least one strain of probiotic bacteria belongs to the species Lactobacillus rhamnosus (LGG).

In an even more preferred embodiment of the present invention the at least one strain of probiotic bacteria is a Lactobacillus rhamnosus (DSM 33870), a mutant thereof and a variant thereof.

Surprisingly, the viability of the bacteria produced by the method of the present invention is particularly high and will stay high after a storage time of several months at a temperature of 4-10° C. The viability of the at least one strain of bacteria in one embodiment is at least 1% of the initial CFU/ml after storage for at least 30 days at a temperature of 4° C., such as after at least 42 days of storage at a temperature of 4° C., such as after at least 70 days of storage at a temperature of 4° C.

The viability of bacteria is measured by counting techniques known to a person skilled in the art, such as, for example, mass count, surface count, Malassez cells, direct counting, turbidity, nephelometry, electronic counting, flow cytometry, fluorescence, impedimetry and image analysis.

In a preferred embodiment the viability of the at least one strain of bacteria produced with the method of the present invention is at least 30% of the initial CFU/ml, preferably at least 50% of the initial CFU/ml, and more preferred at least 70% of the initial CFU/ml under the above-mentioned conditions of storage.

In a preferred embodiment, the method for producing bacteria of the present invention comprises the steps of:
(a) inoculating bacteria in a medium which medium is acidified until a first pH between 3 and 7 is reached;
(b) cultivating the bacteria in the medium while maintaining the first pH by adding an alkaline solution to the medium;
(c) allowing the medium to acidify until a second pH is reached wherein the second pH is lower than the first pH; and
(d) harvesting the bacteria.

In a preferred embodiment, the method for producing bacteria of the present invention comprises the steps of:
(a) inoculating bacteria in a medium which medium is acidified until a first pH between 4.5 and 5.5 is reached;
(b) cultivating the bacteria in the medium while maintaining the first pH for a time of at least 3 hours by incrementally adding an alkaline solution to the medium while monitoring the pH of the medium;
(c) allowing the medium to acidify until a second defined pH of between 2 and 4.5 is reached; and
(d) harvesting the bacteria.

In a preferred embodiment, the method for producing bacteria of the present invention comprises the steps of:
(a) inoculating bacteria in a medium which medium is acidified until a first pH between 4.5 and 5.5 is reached, wherein the defined pH is reached after not more than 18 hours;
(b) cultivating the bacteria in the medium while maintaining the first pH for a time of at least 3 hours by incrementally adding an alkaline solution to the medium while monitoring the pH of the medium;
(c) allowing the medium to acidify until a second pH is reached of between 2 and 4.5, wherein the medium reaches the second pH after 2 to 10 hours; and
(d) harvesting the bacteria.

In a second aspect, the present invention is directed to bacteria obtainable by the method according to the first aspect of the present invention.

In a preferred embodiment the bacteria are in form of a culture, which is frozen or freeze-dried, preferably frozen.

In a third aspect, the present invention is directed to the use of a culture of a bacteria strain according to the second aspect for the production of a food product.

The food product can be a semi solid food product, such as yoghurts, post pasteurized yoghurts, stirred and drinking yoghurts, ambient stable yoghurts, quarks, or fresh cheese.

In a preferred embodiment, the food product is a beverage. The term “beverage” refers to a liquid which one would reasonably contemplate consuming directly. Beverages according to the present invention may be tea, fermented milk products, fruit beverages, plant-based juices, plant-based yogurts, lemonades, soft drinks, sodas, alcoholic beverages, spring or mineral water to which sugar or flavourings may or may not be added, and mixtures thereof.

The beverage may be a tea. As used herein, the term “tea” relates to any product that may comprise cured leaves of the Cameilla sinesis plant, despite oxidation levels. The definition of “tea” used herein encompasses all six different types of tea, including white, yellow, green, oolong, black and post-fermented tea. The definition of “tea” also includes the combination of Cameilla sinesis plus any other additives to the tea including spices, plants, dried fruits, seeds, plant or fruit extracts, whether combined as whole, partial or pulverized. “Tea” also refers to infusion of fruit or herbs made without the tea plant, such as tisane or herbal infusions, but bearing an implied contrast with “tea” as it is construed here. “Tea” may also include herbal or spice varieties, including, but not limited to spearmint, peppermint leaves, ground ginger and ground orange powder. In general, the term “tea” refers to pulverized ingredients (with or without a Camellia sinesis base), which results in a powder form of tea.

In one embodiment, the beverage may be a black tea, flavored black tea, green tea, flavored green tea, Thai (iced) tea, white tea, red tea, herbal tea, as well as a variety of flavored bubble teas (for example, orange, passion fruit, lychee, mango, papaya, kiwi, taro, chocolate, strawberry, raspberry, red bean, mung bean, wheat germ, honeydew, almond, gingerbread, ginger, peanut, peanut butter, butterscotch, sesame, etc).

In one embodiment, the beverage may be a plant-based yoghurt which is derived substantially from or wholly from non-animal sources, but has color, flavor, nutritional content, mouth-feel, texture and/or other qualities that are similar to those of dairy products.

In one embodiment, the beverage may be a fruit beverage. In the present context the term “fruit beverage” refers to a beverage comprising fruit juice, fruit concentrate and/or fruit puree. The term “fruit beverage” covers “fruit juice”, “fruit drink” and “fruit nectar”. The “fruit beverage” may be either one containing pulp, or one from which the pulp has been removed by such an operation as centrifugation. The fruit beverages may further contain e.g. oat, soy, almond, whey and/or non-fermented milk, e.g. in the form of milk powder.

The fruit beverages suitable for the use in the present invention may e.g. be fruit juices, fruit juices from concentrate, fruit drinks, fruit smoothies, and fruit nectar optionally comprising fruit purees and/or water.

The term “fruit juice” refers to the liquid naturally contained in fruit prepared by mechanically squeezing or macerating fresh fruits without the presence of heat and solvents. The “fruit juice” may consist of juice from one type of fruit or a mixture of more than one type of fruit.

The term “fruit drink” in the present context refers to a beverage having a fruit juice content of between 0 to 29%.

The term “fruit nectar” in the present context refers to a beverage having a fruit juice content of between 30 to 99% fruit juice.

In the present context the term “fruit puree” refers to fruits prepared by grounding, pressing and/or straining into the consistency of a thick liquid or a soft paste without the presence of heat and solvents. “Puree” is made of 100% fruit as opposed to being made from just the juice of the fruit.

The total content of fruit juice and/or fruit puree in the fruit beverage is generally between about 20% to about 99.99% by weight, preferably between about 30% to 95% by weight, more preferably between about 40% to 90% by weight, still more preferably between about 50% to 80% by weight, and most preferably 60% to 70% by weight.

In one preferred embodiment the fruit beverage in whole or in part is made from fruits selected from the group consisting of strawberry, banana, grape, orange, apple, mango, peach, blueberry, pineapple, lime, raspberry and blackcurrant and mixtures thereof.

According to a preferred embodiment of the invention the fruit beverage comprises strawberry puree, banana puree, grape juice, orange juice, mango puree, peach puree and blueberry puree.

According to another preferred embodiment of the invention the fruit beverage comprises pineapple juice, orange juice, banana puree, mango puree and lime juice.

According to yet another preferred embodiment of the invention the fruit beverage comprises raspberry puree, blackcurrant juice, blueberry puree, grape juice, strawberry puree and banana puree.

In a particularly preferred embodiment, the beverage is a probiotic fruit beverage.

In a fourth aspect, the present invention relates to a method for producing a food product comprising the steps of:
(i) producing bacteria according to the method of the first aspect of the invention,
(ii) adding the bacteria to the food product; and
(iii) optionally packaging the food product.

According to an embodiment of the present invention, the food product is conveniently packaged in a sealed package that contains from 10 to 5000 ml of the product, such as from 50 to 1000 ml or from 200 to 1000 ml.

Generally, the food product is inoculated with the at least one strain of bacteria in an amount (initial CFU/ml) of from about 1×10⁴ to 1×10¹⁰ CFU/ml, more preferably in an amount from about 1×10⁵ to 1×10⁹ CFU/ml, and even more preferably in an amount from about 1×10⁶ to 1×10⁸ CFU/ml.

The food product produced, can be packaged. In the present context, the term “packaging” the beverage relates to the final packaging of the beverage to obtain a product that can be ingested by e.g. a person or a group of persons. A suitable package may thus be a bottle or carton or similar, and a suitable amount may be e.g. 10 ml to 5000 ml, but it is presently preferred that the amount in a package is from 50 ml to 1000 ml, such as from 200 ml to 1000 ml.

In a fifth aspect the present invention relates to a food product obtainable by the method according to the fourth aspect of the invention, wherein the at least one strain of bacteria is present in the food product in an amount from 1×10³ to 1×10¹⁰ CFU/ml, 1×10⁴ to 1×10⁹ CFU/ml, or 1×10⁵ to 1×10⁸ CFU/ml after 70 days of storage at a temperature between 2 and 10° C.

Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The present invention is further illustrated by the following non-limiting examples.

### DEPOSIT AND EXPERT SOLUTION

The applicant requests that a sample of the deposited microorganisms stated below may only be made available to an expert, subject to available provisions governed by

Industrial Property Offices of States Party to the Budapest Treaty, until the date on which the patent is granted.

TABLE 1
Deposits made at a Depositary institution having acquired the status
of international depositary authority under the Budapest Treaty on the
International Recognition of the Deposit of Microorganisms for the
Purposes of Patent Procedure: Leibniz Institute DSMZ-
German Collection of Microorganisms and Cell Cultures
Inhoffenstr. 7B, 38124 Braunschweig, Germany.

| Strain | Accession No. | Deposit date |
|---|---|---|
| Lactobacillus rhamnosus (LGG) | DSM 33870 | 2021 May 26 |

As used herein, Lactobacillus rhamnosus is now known as Lacticaseibacillus rhamnosus as described in Zheng et al., Int. J. Syst. Evol. Microbiol. DOI 10.1099/ijsem.0.004107.

### EXAMPLES

### Example 1

### Two-step Fermentation with pH Control 0.01% of 2.0E+11 Lactobacillus rhamnosus (LGG) was inoculated in 2L MRS growth media.

The first step of fermentation was conducted at a first pH at 5.0. The pH was monitored and pH was kept at pH 5.0 by adding NaOH. In order to see how much base the strain could consume in total, a control fermentation (#1) was included. For #1 the pH was kept a pH 5 until no more base was consumed. Nine fermentations (#3-11) were included to test different base addition volumes. For these nine fermentations the pH was kept at 5 until a certain amount of base was added, varying from 15-58 g/L. When the desired amount of base was added the base addition was manually terminated.

The second step of fermentation was conducted without base addition i.e. without pH control and allowed to acidify to a second low pH in order to adapt to low pH values varying from pH 3.9-4.4. A second control fermentation (#2) was included. This fermentation was a simple acidification process in one step without pH control (the original process).

The same temperature (40° C.), a headspace flow of 0.2 N2 L/min, an agitation speed of 300 rpm, NaOH for pH control and an inoculation rate of 0.1% were applied for all fermentations, hence only the base addition setting was varied among the included fermentations.

TABLE 2
Fermentation in 2 L scale

| Sample | 1 | 2 | 3 | 4 | 5 | 6 | 7 | 8 | 9 | 10 | 11 |
|---|---|---|---|---|---|---|---|---|---|---|---|
| Fermentation | | | | | | | | | | | |
| STEP 1 | | | | | | | | | | | |
| pH setpoint | 5 | NA | 5 | 5 | 5 | 5 | 5 | 5 | 5 | 5 | 5 |
| Base used | 69.0 | 0.0 | 15.1 | 25.1 | 35.1 | 43.2 | 45.9 | 49.0 | 52.2 | 55.2 | 58.0 |
| (g/L) | | | | | | | | | | | |
| STEP 2 | | | | | | | | | | | |
| Acidification | NA | From | After | After | After | After | After | After | After | After | After |
| | | start | base | base | base | base | base | base | base | base | base |
| Total FM time | 15.7 | 7.87 | 13 | 14 | 15 | 13.6 | 12.8 | 13.2 | 12.9 | 15.8 | 13.1 |
| (hours) | | | | | | 2 | 5 | 2 | 3 | 2 | 3 |
| Conc. factor | 11.9 | 30.2 | 12.4 | 13.4 | 13 | 12.1 | 11.9 | 12 | 12.1 | 12 | 12 |
| Final starter | | | | | | | | | | | |
| culture | | | | | | | | | | | |
| E+10 CFU/g | 9.00 | 1.70 | 0.81 | 2.20 | 4.00 | 4.50 | 4.10 | 4.20 | 5.40 | 5.30 | 5.30 |
| Yield | 840.3 | 331.1 | 806.5 | 746.3 | 769.2 | 826.4 | 840.3 | 833.3 | 826.4 | 833.3 | 833.3 |
| L/10000 L | | | | | | | | | | | |
| % Yield | 8.40 | 3.31 | 8.06 | 7.46 | 7.69 | 8.26 | 8.40 | 8.33 | 8.26 | 8.33 | 8.33 |

*Measured by flow cytometry

TABLE 3
Fermentation in large scale

| | Sample | | |
|---|---|---|---|
| | 12 | 13 | 14 |
| Fermentation | 650 L | 350 L | 17.000 L |
| STEP 1 | | | |
| pH setpoint | NA | 5 | 5 |
| Base used | NA | 55 | 55 |
| (g/L) | | | |
| STEP 2 | | | |
| Acidification | From start | After base | After base |
| Total FM time | 8.07 | 14.58 | 15.25 |
| (hours) | | | |
| Conc. factor | 30.14 | 11.43 | 12.1 |
| Final starter | | | |
| culture | | | |
| E+10 CFU/g | 1.60 | 6.30 | 7.41 |
| Yield | 331.8 | 874.9 | 826.4 |
| L/10000 L | | | |
| % Yield | 3.32 | 8.75 | 8.26 |

The results show that a higher yield is obtained by controlling the pH. The yield was calculated based on a 10 m³ tank. E.g. #11 had a concentration factor of 12× leading to a yield of 10000/12=833.3 kg/10 m³. In comparison the process without pH control #2 had a concentration factor of 30.2× leading to a yield of 10000/30.2=331 kg/10 m³. Hence the product yield with the new two-step fermentation process is increased with >300%.

A higher yield measured as CFU/g was observed when base was added. When no base was added 1.7E+10 CFU/g was observed (#2), whereas 5.3E+10 CFU/g was observed when 58 g/L base was added (#11). Increasing CFU/g were observed with increasing amounts of base added (#3-11).

### Example 2

### Survival in Juice

Three samples of starter culture were produced according to example 1.

A CFU count of all samples were conducted by plating dilutions of 1:1.00E+7, 1.00E+8, 1.00E+9, 1.00E+10, 1.00E+11, and 1.00E+12 (10\hat{ }7, 10\hat{ }8, 10\hat{ }9, 10\hat{ }10, 10\hat{ }11 & 10\hat{ }12) of each sample on De Man, Rogosa and Sharpe agar (MRS), pH 6.5 (Difco, 288210) and incubate for 24 hours at 37° C.

Starter culture was inoculated in 100 mL Nikoline orange juice (Rynkeby, Denmark) at an inoculation level of 1.00E+6 (1*10\hat{ }6) CFU/mL and stored for 8 weeks at 4° C. All samples were done in duplicate.

After storage CFU count were conducted by plating each sample on MRS agar, pH 6.5, and incubated at 20° C. anaerobically for 48 hours. Samples were stomached before plating for CFU to avoid bacteria aggregates by mixing 5 mL juice with 45 mL peptone.

TABLE 4
Survival in juice

| | 0 weeks | 2 weeks | 4 weeks | 6 weeks | 8 weeks |
|---|---|---|---|---|---|
| | (CFU/mL) | (CFU/mL) | (CFU/mL) | (CFU/mL) | (CFU/mL) |
| Beginning | 1.20E+06 | 1.12E+06 | 1.00E+06 | 9.20E+05 | 5.20E+05 |
| Beginning | 1.20E+06 | 1.28E+06 | 4.00E+05 | 9.60E+05 | 4.40E+05 |
| Average | 1.20E+06 | 1.20E+06 | 7.00E+05 | 9.40E+05 | 4.80E+05 |
| Stddev | 0.00E+6 | 1.13E+05 | 4.24E+05 | 2.83E+04 | 5.66E+04 |
| Middle | 1.40E+06 | 1.12E+06 | 5.40E+05 | 7.20E+05 | 5.60E+05 |
| Middle | 1.20E+06 | 1.32E+06 | 8.00E+05 | 8.00E+05 | 4.60E+05 |
| Average | 1.30E+06 | 1.22E+06 | 6.70E+05 | 7.60E+05 | 5.10E+05 |
| Stddev | 1.41E+05 | 1.41E+05 | 1.84E+05 | 5.66E+04 | 7.07E+04 |
| End | 2.20E+06 | 1.02E+06 | 2.00E+04 | 5.40E+05 | 3.00E+05 |
| End | 2.80E+06 | 7.40E+05 | 5.20E+05 | 7.40E+05 | 4.60E+05 |
| Average | 2.50E+06 | 8.80E+05 | 2.70E+05 | 6.40E+05 | 3.80E+05 |
| Stddev | 4.24E+05 | 1.98E+05 | 3.54E+05 | 1.41E+05 | 1.13E+05 |

Results showed a very good stability over the 8 weeks in the orange juice.

1. A method for producing bacteria comprising the steps of:
(a) inoculating bacteria in a medium which medium is acidified until a first pH between 3 and 7 is reached;
(b) cultivating the bacteria in the medium while maintaining the first pH by adding an alkaline solution to the medium;
(c) allowing the medium to acidify until a second pH is reached wherein the second pH is lower than the first pH; and
(d) harvesting the bacteria.

2. The method according to claim 1, wherein the first pH is between 3.5-6.5.

3. The method according to claim 1, wherein the first pH of the medium in step b) is maintained by incremental addition of alkaline solution while monitoring the pH of the medium.

4. The method according to claim 1, wherein the alkaline solution is an aqueous solution of an alkali metal or alkaline earth metal hydroxide or ammonium hydroxide.

5. The method according to claim 1, wherein the first pH of the medium in step b) is maintained until a desired amount of bacteria is obtained.

6. The method according to claim 5, wherein the first pH of the medium in step b) is maintained for a period of 1-24 hours.

7. The method according to claim 1, wherein the second pH is between 2.0-6.0.

8. The method according to claim 1, wherein the total time of steps a) to c) is between 4-24 hours.

9. The method according to claim 1, further comprising the steps of:
e) concentrating the bacteria; and/or
f) freezing or freeze-drying the bacteria in the presence or absence of a cryoprotecting agent.

10. The method according to claim 1, wherein the bacteria is a probiotic bacteria selected from a Lactobacillus, a Bifidobacterium, or a Oenococcus oeni bacterium.

11. The method according to claim 1, wherein the viability of the bacteria is at least 1% of the initial CFU per ml of a food product after storage at 30 days at 4° C.

12. Bacteria obtained by the method according to claim 1.

13. (canceled)

14. A method for producing a food product comprising the steps of:
(i) producing bacteria according to the method of claim 1,
(ii) adding the bacteria to the food product; and
(iii) optionally packaging the food product.

15. The method according to claim 14, wherein the food product is a beverage is selected from the group consisting of tea, fermented milk products, fruit beverages, plant-based juices, plant-based yogurts, lemonades, soft drinks, sodas, alcoholic beverages, spring or mineral water, and mixtures thereof.

16. A food product obtained by the method according to claim 14, wherein the bacteria is present in the food product in an amount from 1×10³ to 1×10¹⁰ CFU/ml after 70 days of storage at a temperature between 2 and 10° C.
